(19) United States
(12) Patent Application Publication
(10) Pub. No.: US 2023/0082681 A1
(43) Pub. Date: Mar. 16, 2023

(54) PERSONAL COMPOSITE INFORMATION-BASED HEALTHCARE SOLUTION PROVISION METHOD AND SYSTEM

(71) Applicant: CLINOMICS INC., Ulsan (KR)

(72) Inventors: Yun Sung CHO, Yongin-si (KR); Jong Hwa BHAK, Ulsan (KR); Byung Chul KIM, Suwon-si (KR)

(21) Appl. No.: 17/801,241

(22) PCT Filed: Apr. 9, 2020
(86) PCT No.: PCT/KR2020/004830

§ 371 Date: Aug. 20, 2022

(30) Foreign Application Priority Data
Mar. 26, 2020 (KR) 10-2020-0036691

(51) Int. Cl. G16H 20/60; G16H 10/20; G16H 50/30

(52) U.S. Cl. CPC G16H 20/60; G16H 10/20; G16H 50/30

(57) ABSTRACT

The present invention provides a healthcare solution provision method. More specifically, the present invention relates to a composite information-based healthcare solution provision method and system in which the nutritional status of an individual is checked, and genetic information and various relevant composite information are used when prescribing, selling, promoting and suggesting a nutritional supplement to the individual, and thus personalized nutrients, nutritional supplements and food which are optimized for each individual are proposed. According to the present invention, an individual checks a genetic and environmental type thereof, and thus assumes a personal bio-constitution type which is a comprehensive body constitution type, and receives a recommendation for the most optimized nutritional supplement or food, and scientific information enabling increasing the selectivity of the individual is provided, and as a result, the effect of using the personal nutritional supplement is increased, and the advantage is achieved of avoiding the use of unnecessary nutritional supplements or reducing the level of side-effects that may happen.

### TECHNICAL FIELD

The present invention relates to a method for providing a customized healthcare solution and relates to a method and system or providing a healthcare solution, based on personal composite information, which proposes customized nutrients, nutritional supplements, favorite foods, and foods, optimized for each individual, by utilizing the categorized concept of constitution and various information, such as genetic information, epigenetic information, clinical information, medical information, body feature information, lifestyle information, symbiotic microbiome information, microorganisms and body-derived cfDNA (circulating free DNA) in checking a person's nutritional status and prescribing, selling, promoting, and recommending nutritional supplements to individuals.

### BACKGROUND ART

The market for health-related vitamins, minerals, amino acids and probiotics, enzyme preparations, and microorganisms is growing every year. Nutrients are fundamentally necessary for human life and physical activity, and their deficiency causes various physical and physiological health problems. Further, when a specific nutritional component is consumed in excess of food or nutritional supplements, side effects occur.

Currently, the most representative nutritional supplement is a multivitamin that contains various vitamins and minerals. However, depending on health conditions, such as genetic characteristics, lifestyle, and disease for each individual, insufficient nutrients and adequate or sufficient nutrients are present in the body. Further, as nutrients affect the growth of microorganisms in various places in the body, they may have a great effect on disease response ability and health condition.

Therefore, instead of providing a comprehensive nutritional supplement through a standardized combination of nutrients, proposing a combination of nutrients and individual nutrients optimized for each individual's genetic type, environmental situation, clinical information, lifestyle, and individual microbiome types is expected to be able to increase efficiency while reducing side effects.

Accordingly, the present invention proposes a method which is based on comprehensive personal genetic characteristics coming from the genetic information about the individual and the epigenetic genetic information and the genetic information about the individual microorganism from the viewpoint of the genome constitution, secures various health-related information through regular physical examinations, regular/irregular clinical and medical information, lifestyle, and questionnaires for directly or indirectly checking clinical, medical, health and nutritional status information from the environmental constitution point of view, and provides information about nutrients suitable for each individual by fusing the results derived according to the two perspectives and assuming a comprehensive bio constitution for each individual.

### DETAILED DESCRIPTION OF THE INVENTION

### Technical Problems

The present invention aims to provide a method and system for providing a healthcare solution based on composite information to provide health information, which may easily grasp an individual's current health condition by using various information, such as the individual's genetic information, epigenetic information, clinical/medical information, lifestyle, the type, number, and genotype of microorganisms inhabiting the individual's body.

The present invention also aims to provide a method and system for providing a healthcare solution based on composite information, which may propose, to individuals, and allow them to purchase, customized nutrients, nutritional supplements, and food optimized for each individual according to their health information.

### Means to Address the Problems

To achieve the foregoing objectives, according to a preferred embodiment of the present invention, a method for providing a healthcare solution based on composite information about an individual by a healthcare server may comprise the steps of: collecting one or more pieces of composite information including genetic information about a user from a user terminal; obtaining per-nutrient numerical information reflecting the user's current nutritional status by analyzing the composite information; and providing the user terminal with health information which is an analysis result of estimating a plurality of nutrients deficient for the user according to the numerical information.

The genetic information may further include epigenetic information and microbiome information about the user. The step of obtaining the per-nutrient numerical information reflecting the user's current nutritional status by analyzing the composite information may include the steps of deriving a nutrient appropriate for the user's epigenetic type and microbiome type for the epigenetic information and the microbiome information; respectively, and obtaining the per-nutrient numerical information.

The genetic information may further include lifestyle information about the user. The step of obtaining the per-nutrient numerical information reflecting the user's current nutritional status by analyzing the composite information may include the steps of: providing the user terminal with an online questionnaire including a plurality of items for the user's lifestyle related to nutrients; receiving lifestyle information including a questionnaire result from the user terminal; and deriving one or more excessive or deficient nutrients for the user according to the lifestyle information and obtaining the per-nutrient numerical information.

The genetic information may include medical information including a medical checkup or medical examination result for the user. The method may further comprise the steps of providing user information to a designated medical institution server to request the medical information, receiving pre-stored medical information about the user from the medical institution server, and deriving a nutrient appropriate for the user's current health condition according to the medical information and obtaining the per-nutrient numerical information.

The numerical information is based on the individual's composite information calculated by the following equation, when a numerical value for a nutrient is represented as a score from 0 to 100,

$$[ ( {\sum }_{i}^{n} N {}_{i} / S \times n ) \times 100 ]$$

S is a maximum natural number that each nutrient may have, N is a score of a nutrient included in a specific group, and n is the number of nutrients.

The method may further comprise the steps of, after providing the user terminal with the analysis result of estimating the plurality of nutrients deficient for the user according to the numerical information, receiving a nutritional supplement purchase request from the user terminal; and providing nutritional supplement information about each or a combination of a plurality of estimated nutrients according to a result of nutrition analysis for the user to one or more food market servers to perform, by proxy, a nutritional supplement purchase procedure appropriate for the user.

Further, to achieve the foregoing objectives, according to another aspect of the present invention, a system for providing a healthcare solution based on composite information about an individual may comprise one or more user terminals receiving genetic information about a user and a healthcare server connected with the one or more user terminals to collect and analyze the genetic information and providing health information related to each user's health according to a result of the analysis. The healthcare server may include an information collection unit collecting one or more pieces of composite information including genetic information about each user from the one or more user terminals, an information analysis unit analyzing the composite information to obtain per-nutrient numerical information reflecting the user's current nutritional status, an nutrient estimation unit estimating a plurality of nutrients deficient for the user according to the numerical information and providing the current health information about the user according to the analysis result to the user terminal, and a database storing collected user information and analysis result for each user.

The genetic information may further include epigenetic information and microbiome information about the user. The information analysis unit may derive a nutrient appropriate for the user's epigenetic type and microbiome type for the epigenetic information and the microbiome information, respectively, and obtains the per-nutrient numerical information.

The genetic information may further include lifestyle information about the user. The information analysis unit may provide the user terminal with an online questionnaire including a plurality of items for the user's lifestyle related to nutrients, receive lifestyle information including a questionnaire result from the user terminal, and derive one or more excessive or deficient nutrients for the user according to the lifestyle information and obtaining the per-nutrient numerical information.

The genetic information may include medical information including a medical checkup or medical examination result for the user. The healthcare server may further include a medical institution association unit providing user information to a designated medical institution server to request the medical information, receiving pre-stored medical information about the user, providing the medical information to the information analysis unit to derive a nutrient appropriate for the user's current health condition, and allowing the per-nutrient numerical information to be applied to the health information.

The healthcare server may further include a product order unit, upon receiving a nutritional supplement purchase request from the user terminal, providing nutritional supplement information about each or a combination of a plurality of estimated nutrients according to a result of nutrition analysis for the user to one or more food market servers to perform, by proxy, a nutritional supplement purchase procedure appropriate for the user.

The user terminal may include a terminal communication unit performing data communication with the healthcare server through an information communication network, a genetic information input unit receiving genetic information according to a genetic test performed on a user and providing the genetic information to the healthcare server, an epigenetic information input unit receiving microbiome information or epigenetic information according to one or more of an epigenetic test and microbiome test performed on the user and providing the epigenetic information and the microbiome information to the healthcare server, an other information generation unit providing information about one or more of lifestyle information input by the user and questionnaire information including the user's response to an electronic questionnaire provided from the healthcare server, a health information display unit receiving health information about the user, provided from the healthcare server and displaying the health information on the screen, and a storage unit cumulatively storing health information received per date.

The user terminal may further include a market use unit performing an order procedure for a nutritional supplement for a necessary nutrient derived according to current health information, by the user's selection.

### Effects of the Invention

The present invention may assume an individual bio composition which is a comprehensive constitution by identifying the individual's genetic and environmental types, provide scientific information to recommend the most optimized nutritional supplement or food and increase the individual's selection, increase the effect of use of the nutritional supplement as a result, and avoid use of unnecessary nutritional supplements or reduce side effects.

### BRIEF DESCRIPTION OF THE DRAWINGS

FIG. 1 is a view schematically illustrating an overall structure of a system for providing a healthcare solution based on composite information about an individual according to an embodiment of the present invention.

FIG. 2 is a view schematically illustrating a structure of a user terminal of a system for providing a healthcare solution based on composite information about an individual according to an embodiment of the present invention.

FIG. 3 is a view schematically illustrating a structure of a healthcare server of a system for providing a healthcare solution based on composite information about an individual according to an embodiment of the present invention.

FIG. 4 is a view schematically illustrating a method for providing a healthcare solution based on composite information about an individual according to an embodiment of the present invention.

### BEST MODE TO PRACTICE THE INVENTION

Throughout the specification, when an element “includes” another element, the element may further include the other element, rather excluding the other element, unless particularly stated otherwise. Further, the term “unit,” “server,” or “system” as used herein denote a unit processing one or more functions or operations and be implemented in hardware, software, or a combination thereof.

Although some exemplary embodiments of the disclosure are described herein, the technical spirit or scope of the disclosure are not limited thereto. As used herein, the terms “including,” “having,” or other similar terms are open-ended transition words that do not exclude any additional or other components when used in the claims and may be comprehensively used in a similar manner to that of “comprising.”

Various schemes or methods described herein may be implemented in hardware, software, or a combination thereof. As used herein, the term “unit,” “server,” or “system” may also be equivalent to a computer-related entity, a hardware component, a software component, or a combination thereof. Each function executed on the program of the present invention may be configured as a module unit and may be recorded in one physical memory or be distributed and recorded between two or more memories and recording media.

In the following description, “system for providing a healthcare solution based on composite information about an individual” may be simply referred to as a “healthcare solution providing system” or “system” for convenience of description.

Hereinafter, a system and method for providing a healthcare solution based on composite information about an individual according to an embodiment of the present invention is described with reference to the drawings.

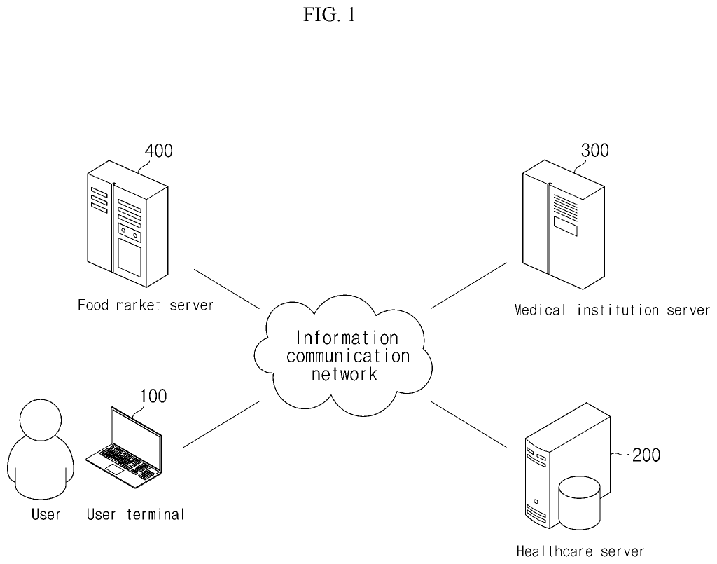

FIG. 1 is a view schematically illustrating an overall structure of a system or providing a healthcare solution based on composite information about an individual according to an embodiment of the present invention.

Referring to FIG. 1, a system for providing a healthcare solution based on composite information about an individual, according to an embodiment of the present invention, may include one or more user terminals 100 receiving genetic information about a user and a healthcare server 200 connected with the one or more user terminals 100 to collect and analyze the genetic information and provide health information related to each user's health according to a result of the analysis. The healthcare server 200 may be connected with an external medical institution server 300 providing medical information related to the user and a food market server 400 for ordering and selling, online, commercial goods, such as nutritional supplements and health functional foods, according to the type of the service provided to the user.

The user terminal 100 may be a computing device, such as a smartphone or a tablet PC possessed by an individual, capable of data communication, and run a web browser or a dedicated application to allow the user to input his genetic information according to his test or to generate questionnaire information in response to a health-related questionnaire and provide it to the healthcare server 200 through an information communication network.

Accordingly, the healthcare server 200 may analyze the collected genetic information about users to identify the current health condition of the user and generate health information including information about nutrients that are deficient or need supplementation and provide the generated health information to the user terminal 100. The user terminal 100 may receive and display the health information on the screen so that the user may identify his current status and information related to future healthcare.

As the user terminal 100, all types of handheld-based mobile terminal devices, such as PCS(Personal Communication System), GSM(Global System for Mobile communications), PDC(Personal Digital Cellular), PHS(Personal Handyphone System), PDA(Personal Digital Assistant), IMT(International Mobile Telecommunication)-2000, CDMA(Code Division Multiple Access)-2000, W-CDMA(W-Code Division Multiple Access), Wibro(Wireless Broadband Internet) terminal, smartphone, smartpad, or tablet PC, and stationary PCs, laptop computers, or other computing devices may be used.

The healthcare server 200 may collect various types of information for identifying the nutritional statuses of individuals, such as epigenetic information, lifestyle information, clinical/medical information, and questionnaire information, as well as genetic information about users from a plurality of user terminals 100 through the information communication network, informing the user of the current health condition by systematically analyzing the innate genetic status and current nutritional status according to the genetic constitution, environmental constitution, and bio constitution which is a fused form of the genetic constitution and the environmental constitution, which may be the individual's own scientific constitutions, and suggest nutrients, nutritional supplements, and customized foods further optimized for the individual.

To provide these functions, the healthcare server 200 may collect, compile or integrate, and analyze genetic and epigenetic information including various omics information, such as the user's genome, epigenome, transcriptome, proteome, metabolome and microbiome, medical information by medical examinations, such as health checkups and specific medical tests, and lifestyle information by a questionnaire including eating habits, drinking, smoking, weight, whether healthcare is executed, and whether he has disease, to identify and predict the individual's nutritional status.

The above-described information may be technically divided into a genetic constitution, an environmental constitution, and a bio constitution that integrate the genetic constitution and the environmental constitution. The healthcare server 200 may determine the individual's type and perform advanced personalized constitution information analysis according to an integrated classification system obtained by systematizing and categorizing the types of data.

The conventional healthcare service platform simply provides information regarding vitamins or health functional foods which are widely sold and known, rather than being based on precise scientific-based genotyping results in identifying the individual's nutritional status and prescribing, selling, promoting, and recommending nutritional supplements to the individual. Of course, such method may also present a certain level of effect.

However, as there is clearly a difference according to the environment and the change of genetic information due to genetic differences and acquired lifestyles for each individual, the healthcare server 200 according to an embodiment of the present invention may overcome the limitations of the conventional methods and systematically identify and classify the individual's innate genetic status and current nutritional status according to the individual's scientific constitutions, which are genetic constitution and environmental constitution and the bio composition which is a fusion of the genetic constitution and environmental constitution, by comprehensively utilizing the individual's genetic information, epigenetic information, clinical information, medical information, lifestyle information, microbiome information about various microbiomes inhabiting the individual's body, and questionnaire information that the individual provides.

Meanwhile, as information communication networks used for connection between the healthcare server 200 and the terminal or between other servers, LTE (long-term evolution), LTE-A (LTE Advance), CDMA (code division multiple access), WCDMA (wideband CDMA), universal mobile telecommunications system (UMTS), wireless broadband (WiBro), or global system for mobile communications (GSM) may be used.

The medical institution server 300 serves to provide medical information among the health-related information about the user that the healthcare server 200 collects. The user who uses the service of the present invention may provide medical information including medical examinations, such as health checkups and medical tests, through the medical institution to the healthcare server 200 and, to that end, request the medical institution server 300 to provide his own medical information, registered in the medical institution server 300, to the healthcare server 200.

Accordingly, the medical institution server 300 may transmit medical information requested by the user to provide to the healthcare server 200 among the registered medical information, to the healthcare server 200.

The food market server 400 may operate one or more online markets that sell various health-related products, such as nutritional supplements and health functional foods. The user may identify the health information provided by the healthcare server 200, order nutritional supplements directly from the online market through his user terminal 100, or order appropriate nutritional supplement products based on the registered health information through the product purchase agency service provided by the healthcare server 200.

According to the above-described agency service, the healthcare server 200 may order nutritional supplements for nutrients that are deficient or need supplementation for the user according to the generated health information, with the user set as the recipient, The food market server 400 may identify the order, select an appropriate product, process the order process, and ship the product to the user.

Here, the products carried by the food market server 400 may include not only nutritional supplement and health functional foods but also general food, such as lunch boxes capable of supplying necessary nutrients.

According to the above-described structure, in the system according to an embodiment of the present invention, a plurality of users may create a database of genetic information using their user terminal, and the healthcare server may collect, online, and analyze various information, such as genetic information, epigenetic information, lifestyle information, and questionnaire information input and stored in the user terminal to thereby determine the current nutritional status of each user, and provide the result, as health information, to the user terminal, so that the user may easily obtain information about his current health condition and information necessary for future healthcare even without visiting a medical institution.

The healthcare server included in the system for providing a healthcare solution according to an embodiment of the present invention is described below in detail with reference to the drawings.

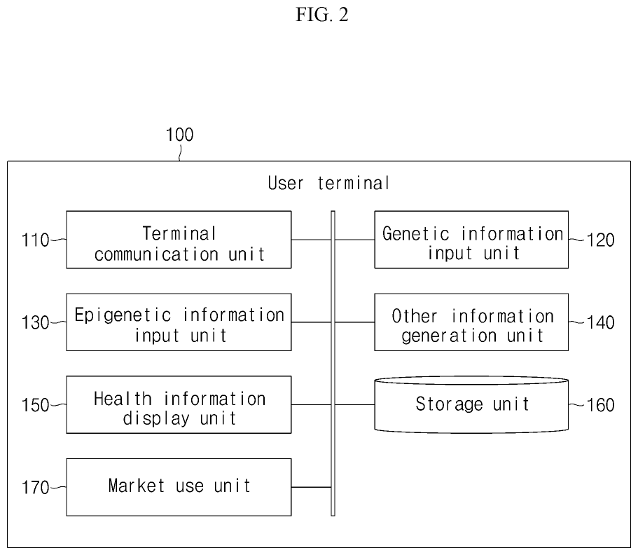

FIG. 2 is a view schematically illustrating a structure of a user terminal of a system for providing a healthcare solution based on composite information according to an embodiment of the present invention. In the following description, the user terminal and each component constituting the user terminal may be implemented in the form of a computer program executable by a known microprocessor and be recorded on a readable and writable recording medium and be mounted on a mobile device.

Referring to FIG. 2, according to an embodiment of the present invention, the user terminal 100 may include a terminal communication unit 110 performing data communication with the healthcare server through an information communication network, a genetic information input unit 120 receiving genetic information according to a genetic test performed on a user and providing the genetic information to the healthcare server, an epigenetic information input unit 130 receiving microbiome information or epigenetic information according to one or more of an epigenetic test and microbiome test performed on the user and providing the epigenetic information and the microbiome information to the healthcare server, an other information generation unit 140 providing information about one or more of lifestyle information input by the user and questionnaire information including the user's response to an electronic questionnaire provided from the healthcare server, a health information display unit 150 receiving health information about the user, provided from the healthcare server and displaying the health information on the screen, a storage unit 160 cumulatively storing health information received per date, and a market use unit 170 performing an order procedure for a nutritional supplement for a necessary nutrient derived according to current health information, by the user's selection.

The terminal communication unit 110 may be connected with the healthcare server through the information communication network to perform data communication for performing the healthcare service of the present invention. Upon connection with the healthcare server, the terminal communication unit 110 may provide, through data communication, various composite information related to the user's current bio composition, such as the genetic information, epigenetic information, microbiome information, lifestyle information, and questionnaire information about the user, stored in the storage unit 160 and may receive the result of analysis from the healthcare server.

The genetic information input unit 120 may receive information related to the user's genetic information among the composite information. Genetic information is information that targets various genetic mutations, epigenetic mutations, and expression level mutations that are related to nutrients and may be used to calculate each individual's per-nutrient values by a known genetic test.

The epigenetic information input unit 130 may store the genetic information input by the user in the storage unit 160 and may provide the genetic information to the healthcare server through the terminal communication unit 110.

The epigenetic information input unit 130 may receive information related to the user's epigenetic information among the composite information. The epigenetic information relates to the characteristics expressed with respect to genes by the interaction between genetic factors in the normal development process, may be expressed by the internal factors, such as genetic factors and hormone, and external factors, such as life pattern and eaten foods, and may include changes in nutrition and growth. Such epigenetic information may be tested in the same manner as is performed on the genetic information. The epigenetic information input by the user may be stored, and provided to the healthcare server, by the epigenetic information input unit 130.

The other information generation unit 140 may receive other various composite information about the user in addition to the above-described genetic information and epigenetic information.

The other composite information includes information about the microbiome that inhabits the individual's body. The microbiome information may be obtained simultaneously with, or separately from, the test for obtaining the above-described genetic information and may include information known to be related to the nutrient or food fitting the individual's microbiome type or appearing to be predicted or inferred by the gene type.

The other composite information also includes lifestyle information. Such lifestyle information may be obtained through a questionnaire on items that are related to or have a causal relationship with nutrients.

The other composite information also includes clinical and medical information. The medical information may be information obtained through a medical test, identified in the hospital or a specific test or checkup. The user himself may input his own clinical or medical information through the user terminal and provide it to the healthcare server or according to an agreement between medical institutions, the medical institution server may provide the user's medical information, instead of the user, to the healthcare server.

The health information display unit 150 may receive the health information according to the analysis which is based on the composite information provided to the healthcare server by the user terminal 100 and display the health information on the screen of the user terminal 100.

The storage unit 160 may store various data necessary for using the service. The data stored in the storage unit 160 may include the above-described genetic information and epigenetic information and the health information provided by the healthcare server, and the storage unit 160 stores, by date, the information in the storage unit 160. Further, the storage unit 160 may further store account information for accessing the healthcare server.

Meanwhile, the user terminal 100 according to an embodiment of the present invention may provide the function of allowing the user to order and purchase, from an online market, a nutritional supplement based on the health information, in addition to the function of allowing the user himself to identify the health information by simply displaying the health information on the screen.

As a component therefor, the user terminal 100 may further include the market use unit 170 that performs the order procedure for the nutritional supplement for supplementing the necessary nutrient, derived according to the current health information, by the user's selection. The market use unit 170 may order and request a health functional food according to the health information, from the online market run by the food market server or request the healthcare server to order a health functional food currently suitable for the user.

The healthcare server of the system for providing a healthcare solution based on composite information according to an embodiment of the present invention is described below in detail with reference to the drawings.

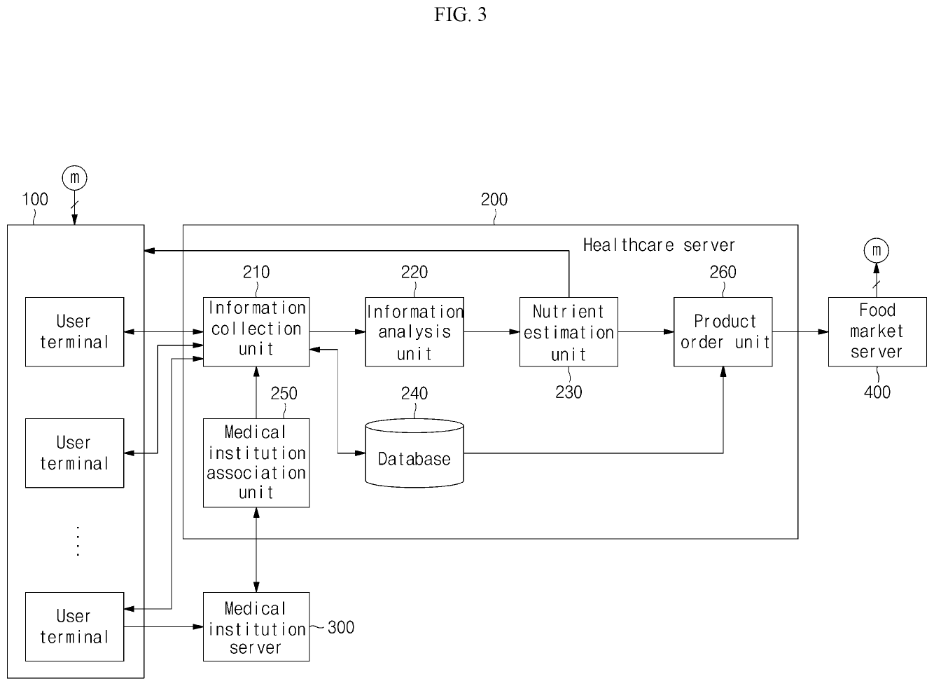

FIG. 3 is a view schematically illustrating a structure of a healthcare server of a system for providing a healthcare solution based on composite information according to an embodiment of the present invention.

Referring to FIG. 3, according to an embodiment of the present invention, the healthcare server 200 may include an information collection unit 210 collecting one or more pieces of composite information including genetic information about each user from one or more user terminals 100, an information analysis unit 220 analyzing the composite information to obtain numerical information per nutrient reflecting the current nutritional status for the user, an nutrient estimation unit 230 estimating a plurality of nutrients deficient for the user according to the numerical information and providing the current health information about the user according to the analysis result to the user terminal 100, a database 240 storing collected user information and analysis result for each user, a medical institution association unit 250 providing user information to a designated medical institution server 300 to request medical information and receive pre-stored medical information about the user, providing the medical information to the information analysis unit 220 to derive a nutrient appropriate for the user's current health condition, and allowing the per-nutrient numerical information to be reflected in the health information, and a product order unit 260 that, upon receiving a nutrient purchase request from the user terminal 100, provides nutrient information about the estimated individual nutrients or a combination thereof to one or more food market servers to perform a nutritional supplement purchase procedure appropriate for the user, instead of the user.

The information collection unit 210 may be connected with one or more user terminals through the information communication network to collect various types of information, i.e., composite information, for determining each user's health condition. The composite information may include genetic information according to a genetic test on the user, and epigenetic information, microbiome information, and lifestyle information.

The information analysis unit 220 may analyze the composite information collected from the user terminal 100, analyzing and predicting each user's current health condition.

Specifically, the information analysis unit 220 may collect, integrate, and analyze the composite information in various points of view, such as genetic information, epigenetic information, medical information, and lifestyle information, to identify and predict the user's nutritional status. Such information is divided into the genetic constitution, environmental constitution, and the bio composition which is a fusion of the genetic constitution and the environmental constitution, according to the field, and the information analysis unit 220 may systematize the data types, determine each individual's type according to the integrated classification system, perform advanced personalized constitution information analysis corresponding thereto, and output the result as numerical information.

First, when the analysis target is genetic information, the per-nutrient values for each individual may be calculated through a genetic test, targeting various genetic mutations, epigenetic mutations, and expression level mutations that are related to nutrients, in this case, a specific epigenetic or genetic marker is not specified.

For example, an analysis algorithm using a genetic test performs a genetic test based on information related to the levels of various vitamins, minerals, amino acids, proteins, fatty acids, coenzymes, carotenoids, collagen, and probiotics, thereby determining whether the levels of the nutrients genetically owned by the individual are high or low. The analysis of the genetic test results may not only suggest genetical per-nutrient statuses of each individual but also derive information, such as the overall result of related nutrients per function, per-nutrient definitions, symptoms in case of deficiency, side effects in case of overdose, recommended daily amount, foods rich in nutrients, good and bad nutrients when consumed together, and nutritional supplement intake times.

In the same manner as the genetic test, the epigenetic test may be performed simultaneously with or separately from the genetic test and, based on the epigenetic information, it is possible to derive information that may be inferred or predicted according to the gene type or is known to be related to foods or nutrients appropriate for the individual's epigenetic type in the same manner as the result of genetic test based on the epigenetic information.

The same method as the genetic test or epigenetic test may be performed on the microbiome information simultaneously therewith, and the microbiome information may indicate information about the type of microbiome inhabiting the individual's body, thereby deriving information related to the nutrition appearing to be inferred and predicted according to the gene type or known to be related for the nutrient or food appropriate for the individual's microbiome type.

In particular, according to the analysis of microbiome information, if there is a study result that certain bacteria and bacterial groups are above a certain ratio in the intestine, or that certain body enzymes or genes are stimulated to cause a deficiency in a specific nutritional status, it is preferable to prescribe probiotics that may inhibit or encourage the microorganism group, so probiotics or other nutritional supplements may be recommended according to the genetic constitution having information, such as individual's genetic information or epigenetic information. This presents a significant difference in effectiveness from simply providing general probiotics as a nutritional supplement without considering the user's microbial type.

Further, the information analysis unit 220 according to an embodiment of the present invention may analyze the user's health condition through information according to other points of view, as well as the above-described genetic information, epigenetic information, and microbiome information.

As such other information, the lifestyle information is information obtained by a questionnaire regarding the items related to or having a causal relationship with nutrients. The information collection unit 210 may provide an online, electronic questionnaire including a plurality of items in various formats, to the user terminal 100 and receive questionnaire information, and the information analysis unit 220 may analyze the questionnaire information to infer nutrients insufficient for the user.

For example, nutrient components may be derived from dietary habits, such as diet included in the questionnaire information, and the nutritional status relative to the recommended daily amount may be analyzed. Accordingly, it is possible to derive one or more excessive or deficient nutrients for the user through the lifestyle information. The lifestyle is part of an individual's environmental constitution.

Further, clinical and medical information may be information obtained through medical examinations, specific tests, and medical tests identified in the hospital according to the onset of a disease, and it is possible to analyze the nutritional status from blood sugar and blood pressure which are various clinical and medical information values and the user's direct nutritional status. For example, when the user's blood pressure and blood sugar are measured as higher than normal values, it is possible to derive nutrients that help reduce blood pressure and blood sugar levels. Such clinical and medical information is part of an individual's environmental constitution.

The information analysis unit 220 according to an embodiment of the present invention may analyze various types of composite information described above and represent the result as a numerical value.

The nutrient estimation unit 230 may estimate a plurality of nutrients deficient for the user according to the numerical information calculated by the above-described information analysis unit 220 and may provide current health information about the user according to the analysis result to the user terminal 100.

Specifically, the nutrient estimation unit 230 may analyze the integrated state of the genetic constitution, which is influenced by the individual's congenital inheritance, and the environmental constitution, which is significantly influenced by the current lifestyle and the environment he faces, through the above-described series of analysis methods to infer the current nutritional status and the cause for the current nutritional status, extract the individual's bio composition, and generate customized nutritional supplement proposal information according thereto.

Here, it may be seen that the individual's bio constitution comes from genetic information, not a subjective and abstract concept, such as one from oriental medicine, and the type is not determined but is customized according to the individual situation.

Accordingly, the nutrient estimation unit 230 according to an embodiment of the present invention may derive the individual's nutritional status based on the numerical information calculated by the information analysis unit 220. In this case, the genetic information, medical information, and lifestyle information may be classified into the genetic constitution, environmental constitution, and bio composition which is a combination of the genetic constitution and the environmental constitution, and may be used for nutrient estimation.

Alternatively, the bio constitution may be finally derived, or each single type of constitution information may be used as the health information. As an example, if the user has a genetic constitution that is predicted to have a lack of concentration, a nutritional supplement known in an animal or human clinical test as helpful in enhancing concentration may be suggested to the user in a customized manner.

Further, the nutrient estimation unit 230 may additionally derive necessary nutrients generally recommended for a specific age group, even when it is not determined that a predetermined nutrient is insufficient for the user according to the result by the information analysis unit 220. For example, in the case of a menopausal woman, if she is not deficient in nutrients according to the numerical information, but are not in an excessive intake state, good nutrients for menopausal women may be additionally presented to her.

Further, the nutrient estimation unit 230 may generate health information including recommendations for maintaining a current good eating habit and nutritional status even for items having a currently recommended nutritional status. Further, when receiving a request for the current status from the user terminal 100, the nutrient estimation unit 230 may add the individual's desired nutrients to the health information. For example, to an examinee who wants to enhance learning ability and concentration, nutrients having a corresponding function may be additionally presented.

Further, in the process of inferring the individual's nutritional status, the nutrient estimation unit 230 may present a result in the form of individually listing the nutrients belonging to the same category group, e.g., vitamins A, B, C, D, E, and K or may combine nutrients having the same or similar functions among several nutrients belonging to different category groups to predict the health and function status and reflect it to the health information.

Table 1 below exemplifies nutrients included in each category.

TABLE 1

| Category | Nutrient |
|---|---|
| vitamin | vitamin A1 (retinol) |
| | vitamin B1 (thiamine) |
| | vitamin B2 (riboflavin) |
| | vitamin B3 (Niacin) |
| | vitamin B5 (pantothenic acid) |
| | vitamin B6 (pyridoxine) |
| | vitamin B7 (Biotin) |
| | vitamin B9 (folic acid) |
| | vitamin B12 (Cobalamin) |
| | vitamin C |
| | vitamin D |
| | vitamin E |
| | vitamin K |
| | choline |
| | inositol |
| mineral | calcium |
| | iron |
| | zinc |
| | potassium |
| | magnesium |
| | copper |
| | chrome |
| | selenium |
| | manganese |
| | iodine |
| | molybdenum |
| amino acid | histidine |
| | leucine |
| | isoleucine |
| | lysine |
| | methionine |
| | phenylalanine |
| | threonine |
| | tryptophan |
| | valine |
| | arginine |
| | alanine |
| | asparagine |
| | aspartic acid |
| | glutamine |
| | glutamic acid |
| | glycine |
| | tyrosine |
| | proline |
| | serine |
| | taurine |
| | betaine |
| | carnitine |
| | carnosine |
| | citrulline |
| | ornithine |
| | creatine |
| fatty acid | omega-3 fatty acids (DHA) |
| | omega-3 fatty acids (EPA) |
| | omega-3 fatty acids (alpha-linolenic acid) |
| | omega-6 fatty acids (linoleic acid) |
| | omega-6 fatty acids (gamma-linolenic acid) |
| | omega-6 fatty acids (arachidonic acid) |
| | omega-7 fatty acids (palmitoleic acid) |
| | omega-9 fatty acids (oleic acid) |
| carotenoid | beta carotene |
| | lutein and zeaxanthin |
| | astaxanthin |
| coenzyme | coenzyme Q10 |
| protein | albumin |
| related to probiotics | lactobacillus, bifidobacterium, etc. |
| | prebiotics |
| collagen-related | collagen |
| | chitosan |
| | chitin |

Meanwhile, the nutrient estimation unit 230 may define nutrients associated with the same or similar health and functions through information, such as definitions, functions, symptoms in case of deficiency, and side effects in case of overdose of various nutrients included in the service. For example, nutrients helpful to eye health may be defined as vitamin A1 in the vitamin group, omega-3 fatty acids (DHA, EPA) in the fatty acid group, beta-carotene, lutein, and zeaxanthin in the carotenoid group.

Table 2 below illustrates helpful nutrients in relation to eye health.

TABLE 2
eye health

vitamin A1
omega-3 fatty acids (DHA)
omega-3 fatty acids (EPA)
beta carotene
lutein and zeaxanthin

Table 3 below illustrates related nutrients per health and function.

TABLE 3
brain health

vitamin B9
vitamin B12
choline
iron
valine
tyrosine
glutamine
omega-3 fatty acids (DHA)
omega-6 fatty acids (arachidonic acid)
cardiovascular health
vitamin B3
vitamin B9
inositol
iron
omega-3 fatty acids (EPA)
omega-3 fatty acids (alpha-linolenic acid)
omega-6 fatty acids (gamma-linolenic acid)
omega-9 fatty acids (oleic acid)
coenzyme Q10
liver health
betaine
choline
inositol
leucine
isoleucine
alanine
ornithine
omega-3 fatty acids (DHA)
albumin
bone and joint health
vitamin A1
vitamin C
vitamin D
vitamin K
calcium
zinc
manganese
energy metabolism
vitamin B1
vitamin B3
vitamin B5
vitamin B6
isoleucine
glutamic acid
omega-3 fatty acids (alpha-linolenic acid)
omega-6 fatty acids (linoleic acid)
coenzyme Q10
immune function
vitamin C
vitamin E
selenium
histidine
lysine
arginine
aspartic acid
threonine
albumin
antioxidant function
vitamin C
vitamin E
selenium
beta carotene
coenzyme Q10
fatigue management
vitamin B1
vitamin C
taurine
aspartic acid
glutamic acid
coenzyme Q10
albumin
skin health
vitamin A1
vitamin C
vitamin E
serine
proline
omega-6 fatty acids (gamma-linolenic acid)
omega-7 fatty acids (palmitoleic acid)
beta carotene

The nutrient estimation unit 230 may calculate a score and grade for each health and function by integrating the high or low levels of nutrients included for each defined health and function. The score for each health and function may be calculated by Equation 1 below.

$$[ ( {\sum }_{i}^{n} N {}_{i} / S \times n ) \times 100 ] \qquad [ 97 ] [ Equation$$

Here, ‘S’ is the maximum score that each nutrient may have and is a natural number, ‘N’ is the score of the nutrient included in a specific health and functional group, and ‘n’ is the number of nutrients included in the corresponding health and function. (For example, if the high and low values for each nutrient are divided into 4 levels, ‘S’ has a value of 4, which is the maximum value in the range of 1, 2, 3, and 4).

According to Equation 1 above, e.g., if, for eye health, the value of vitamin A1 is 1 point, the level of DHA is 3 points, the level of EPA is 0 points, the level of beta carotene is 1 point, and the levels of lutein and zeaxanthin are 2 points, it may be calculated as ‘[(1+3+0+1+2)/(3×5)]×100=46.7’.

In this case, the calculated score may be presented as health information as it is, or the score may be presented as a grade through additional grouping. According to Equation 1 above, since the minimum score for each health and function is 0 points and the maximum score is 100 points, 75-100 points may be classified as A grade, 5075 points as B grade, and 25-50 points as C grade, and 0-25 points as D grade. The criteria for grading and the number of grades presented here are not limited.

The nutrient estimation unit 230 may provide the derived nutrients individually or in combination, along with scores and grades for each health and function.

The database 240 may store the collected user information and analysis results for each user. Further, the database 240 may store member information about users who want to use the health management service provided by the system according to the present invention and the composite information collected for each user and may store, per date, the health information generated according to the update of the composite information.

Further, information about nutrients for health and function shown in Tables 2 and 3 above may be stored in the database 240 and may be referenced when generating health information.

Meanwhile, the healthcare server 200 according to an embodiment of the present invention provides may further include the medical institution association unit 250 for collecting clinical and medical information according to the treatment and examination performed at a medical institution, such as a hospital, for the user, in addition to the genetic information and questionnaire information collected from the user terminal 100 for information analysis.

The medical institution association unit 250 may be connected to the medical institution server 300 through the information communication network and may receive the user's medical information from a designated medical institution server 300 for which information provision is approved by the user. The received medical information may be provided to the information analysis unit 220 and may be used to derive nutrients suitable for the user's current health condition through the above-described analysis step and to reflect numerical information for each nutrient in the health information.

Further, according to an embodiment of the present invention, the user who has received health information may purchase nutritional supplements, health functional foods, etc. as an active action for supplementing deficient nutrients according to the health information. In particular, the healthcare server 200 may provide a food purchase agency service in association with an online food market.

To that end, the healthcare server 200 may further include a product order unit 260 that provides nutrient information about each or a combination of a plurality of estimated nutrients included in the health information provided to the user to one or more food market servers and, by proxy, performs the nutrient purchase procedure suitable for the user.

According to an embodiment of the present invention, the nutrient estimation unit 230 may derive a nutrient combination capable of exhibiting an optimal effect by utilizing nutrient combination information that has a synergistic effect when ingested together.

For example, based on the information that iron absorption increases when vitamin C and vitamin E are taken together with iron, the nutrient estimation unit 230 may provide nutrients tailored to each individual and recommend corresponding nutritional supplements. The product order unit 260 may request the food market server 400 of the online market that sells the recommended nutritional supplement according to the users request, processing the ordering procedure m of the corresponding nutritional supplement.

In particular, when efficacy decreases if the nutrient analyzed to be needed for the individual based on the health information is taken together, e.g., if copper absorption is decreased when copper and vitamin C are taken together, the product order unit 260 may consider such fact and provides a combination of nutritional supplements, as ordered, as a separate product in individual packages or different configurations, to allow the user to take the nutritional supplements with a time gap and process the ordering procedure m.

Further, the associated food market server 400 may provide information, such as foods or ingredients containing sufficient nutrients required for the individual in addition to nutritional supplements and health functional food to the user terminal 100 or sell them through the food market server 400. In this case, in addition to the message transmission to the user terminal 100 when providing information, a wire or verbal consultation by a person in charge may also be used, but embodiments of the disclosure are not limited to a specific method. The food market server 400 may operate various types of online food markets, such as customized restaurants that provide nutritional supplements, ingredients, and food, lunch boxes, and delivery services and may provide a service for selling health-related products.

Hereinafter, a method for providing a healthcare solution based on composite information by a system according to an embodiment of the present invention is described with reference to the drawings.

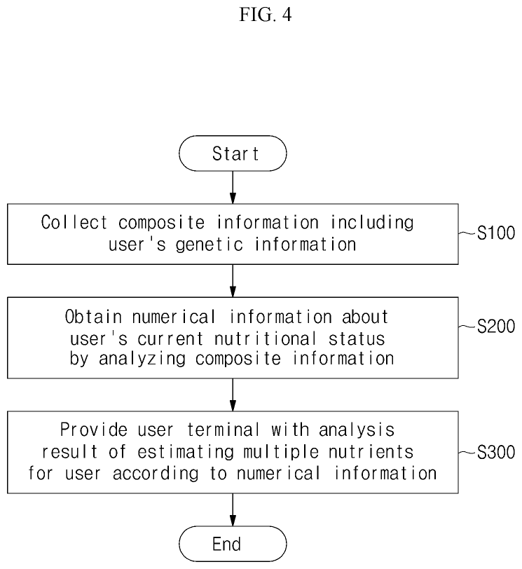

FIG. 4 is a view schematically illustrating a method for providing a healthcare solution based on composite information according to an embodiment of the present invention, Unless specifically stated, each step described below is performed by the above-described healthcare server or its components according to the present invention.

Referring to FIG. 4, according to an embodiment of the present invention, a method for providing a healthcare solution based on composite information may include the step S100 of collecting one or more pieces of composite information including genetic information about a user from a user terminal, the step S200 of obtaining numerical information per nutrient reflecting a current nutritional status for the user by analyzing the composite information, and the step S300 of providing the user terminal with health information which is an analysis result of estimating a plurality of nutrients deficient for the user according to the numerical information.

In the step S100 of collecting one or more pieces of composite information including the genetic information about the user from the user terminal, if the user of the service of the present invention inputs composite information including the genetic information to his user terminal, a healthcare server connected to the user terminal through the information communication network collects composite information per user. Further, in step 3100, the healthcare server may collect epigenetic information, microbiome information, and questionnaire information about life patterns in addition to the genetic test results, such as the user's genome and apply an appropriate analysis algorithm according to the collected information, determining the nutrients currently deficient or excessively taken by the user.

Next, in the step S200 of obtaining the numerical information for each nutrient that reflects the current nutritional status for the user by analyzing the composite information, the healthcare server may analyze the user's current nutritional status through the collected composite information and quantify the result.

In the numerical information according to the analysis, it is possible to calculate the score and grade for each health and function by integrating the high and low levels of nutrients included per health and function defined in the server and be calculated by Equation 1 above.

In particular, when the healthcare server uses epigenetic information and microbiome information as analysis data in addition to genetic information, the healthcare server may derive nutrients suitable for the user's epigenetic type and microbiome type and calculate numerical information for each nutrient.

Further, when using lifestyle information about the user, the healthcare server may provide the user terminal with an online questionnaire including a plurality of items about the user's lifestyle related to nutrients and may receive the lifestyle information including the questionnaire result from the user terminal, derive one or more excessive or deficient nutrients for the user according to the lifestyle information, and calculate numerical information for each nutrient.

Further, the healthcare server according to an embodiment of the present invention may derive health information using medical information including a health checkup or medical examination result for the user in addition to the above-described types of composite information and, as a step for this, the healthcare server may provide user information to a designated medical institution server to request medical information, receive the user's pre-stored medical information from the medical institution server, derive nutrients suitable for the user's current health condition according to the medical information, and calculate numerical information for each nutrient.

Next, in the step S300 of providing the user terminal with the health information, which is an analysis result of estimating a plurality of nutrients deficient for the user according to the numerical information, the healthcare server may provide the score calculated per nutrient, as it is, as the health information, to the user terminal or may provide the score as a predetermined number of grades through per-score grouping. Further, the healthcare server may provide the derived nutrients individually or in combination, along with scores and grades for each health and function.

According to the above-described steps, the user may easily identify his or her current health condition and perform healthcare through the necessary nutrition intake.

Further, the user may make a nutritional supplement purchase request to the healthcare server through the user terminal, and the healthcare server may provide nutritional information about each or a combination of a plurality of estimated nutrients to one or more food market servers according to the nutritional analysis result for the user, thereby performing, as a proxy, a nutritional supplement purchase procedure suitable for the user.

The foregoing specific description should be interpreted as an example of a preferred embodiment, rather than as limiting the scope of the present invention. Accordingly, the scope of the disclosure should be defined by the following claims and equivalents thereof, but not by the above-described embodiments.

1. A method for providing a healthcare solution based on composite information about an individual by a healthcare server, the method comprising the steps of:
collecting one or more pieces of composite information including genetic information about a user from a user terminal; obtaining per-nutrient numerical information reflecting the user's current nutritional status by analyzing the composite information; and
providing the user terminal with health information which is an analysis result of estimating a plurality of nutrients deficient for the user according to the numerical information.

2. The method of claim 1, wherein the genetic information further includes epigenetic information and microbiome information about the user, and wherein the step of obtaining the per-nutrient numerical information reflecting the user's current nutritional status by analyzing the composite information includes the steps of deriving a nutrient appropriate for the user's epigenetic type and microbiome type for the epigenetic information and the microbiome information, respectively, and obtaining the per-nutrient numerical information.

3. The method of claim 1, wherein the genetic information further includes lifestyle information about the user, and wherein the step of obtaining the per-nutrient numerical information reflecting the user's current nutritional status by analyzing the composite information includes the steps of: providing the user terminal with an online questionnaire including a plurality of items for the user's lifestyle related to nutrients; receiving lifestyle information including a questionnaire result from the user terminal; and deriving one or more excessive or deficient nutrients for the user according to the lifestyle information and obtaining the per-nutrient numerical information.

4. The method of claim 1, wherein the genetic information includes medical information including a medical checkup or medical examination result for the user, and wherein the method further comprises the steps of: providing user information to a designated medical institution server to request the medical information; receiving pre-stored medical information about the user from the medical institution server; and deriving a nutrient appropriate for the user's current health condition according to the medical information and obtaining the per-nutrient numerical information.

5. The method of claim 2, wherein the numerical information is based on the individual's composite information calculated by equation
$$[ ( {\sum }_{i}^{n} N {}_{i} / S \times n ) \times 100 ]$$
when a numerical value for a nutrient is represented as a score from 0 to 100, wherein S is a maximum natural number that each nutrient may have, N is a score of a nutrient included in a specific group, and n is the number of nutrients.

6. The method of claim 1, further comprising the steps of, after providing the user terminal with the analysis result of estimating the plurality of nutrients deficient for the user according to the numerical information, receiving a nutritional supplement purchase request from the user terminal; and providing nutritional supplement information about each or a combination of a plurality of estimated nutrients according to a result of nutrition analysis for the user to one or more food market servers to perform, by proxy, a nutritional supplement purchase procedure appropriate for the user.

7. A system for providing a healthcare solution based on composite information about an individual, the system comprising:
one or more user terminals receiving genetic information about a user; and
a healthcare server connected with the one or more user terminals to collect and analyze the genetic information and providing health information related to each user's health according to a result of the analysis, wherein the healthcare server includes: an information collection unit collecting one or more pieces of composite information including genetic information about each user from the one or more user terminals; an information analysis unit analyzing the composite information to obtain per-nutrient numerical information reflecting the user's current nutritional status; an nutrient estimation unit estimating a plurality of nutrients deficient for the user according to the numerical information and providing the current health information about the user according to the analysis result to the user terminal; and a database storing collected user information and analysis result for each user.

8. The system of claim 7, wherein the genetic information further includes epigenetic information and microbiome information about the user, and wherein the information analysis unit derives a nutrient appropriate for the user's epigenetic type and microbiome type for the epigenetic information and the microbiome information, respectively, and obtains the per-nutrient numerical information.

9. The system of claim 7, wherein the genetic information further includes lifestyle information about the user, and wherein the information analysis unit provides the user terminal with an online questionnaire including a plurality of items for the user's lifestyle related to nutrients, receives lifestyle information including a questionnaire result from the user terminal, and derives one or more excessive or deficient nutrients for the user according to the lifestyle information and obtaining the per-nutrient numerical information.

10. The system of claim 7, wherein the genetic information includes medical information including a medical checkup or medical examination result for the user, and wherein the healthcare server further includes a medical institution association unit providing user information to a designated medical institution server to request the medical information, receiving pre-stored medical information about the user, providing the medical information to the information analysis unit to derive a nutrient appropriate for the user's current health condition, and allowing the per-nutrient numerical information to be applied to the health information.

11. The system of claim 7, wherein the healthcare further includes a product order unit, upon receiving a nutritional supplement purchase request from the user terminal, providing nutritional supplement information about each or a combination of a plurality of estimated nutrients according to a result of nutrition analysis for the user to one or more food market servers to perform, by proxy, a nutritional supplement purchase procedure appropriate for the user.

12. The system of claim 7, wherein the user terminal includes a terminal communication unit performing data communication with the healthcare server through an information communication network, a genetic information input unit receiving genetic information according to a genetic test performed on a user and providing the genetic information to the healthcare server, an epigenetic information input unit receiving microbiome information or epigenetic information according to one or more of an epigenetic test and microbiome test performed on the user and providing the epigenetic information and the microbiome information to the healthcare server, an other information generation unit providing information about one or more of lifestyle information input by the user and questionnaire information including the user's response to an electronic questionnaire provided from the healthcare server, a health information display unit receiving health information about the user, provided from the healthcare server and displaying the health information on the screen, and a storage unit cumulatively storing health information received per date.

13. The system of claim 7, wherein the user terminal further includes a market use unit performing an order procedure for a nutritional supplement for a necessary nutrient derived according to current health information, by the user's selection.
